(19) United States
(12) Patent Application Publication
(10) Pub. No.: US 2026/0249012 A1
(43) Pub. Date: Aug. 27, 2026

(54) Medicament Delivery Device

(71) Applicant: Genzyme Corporation, Cambridge, MA (US)

(72) Inventors: Alexander Hee-Hanson, Melbourn (GB); Thomas Lever, Melbourn (GB); Michael Parrott, Melbourn (GB); Robert Wilson, Melbourn (GB); Haiming Wu, Weston, MA (US)

(21) Appl. No.: 19/064,354

(22) Filed: Feb. 26, 2025

(51) Int. Cl. A61M 5/20; A61M 5/32

(52) U.S. Cl. CPC A61M 5/2033; A61M 5/3202; A61M 5/3243; A61M 2005/2073

(57) ABSTRACT

A medicament delivery device for injecting medicament is described. The medicament delivery device has a body having a proximal end and an opposed distal end. The device further comprises a needle for injecting medicament. An actuation member is movable relative to the body from a first position to a second position for dispensing medicament from the needle. A locking member is configured to rotate relative to the body from a pre-use position, in which the actuation member is prevented from moving to the second position, to a use position in which the actuation member is permitted to move to the second position. A coupling rotationally couples the locking member and the cap together when a cap is attached to the body, and thereby rotationally couples the locking member to the body, so as to prevent the locking member from rotating from the pre-use position to the use position.

### TECHNICAL FIELD

The present disclosure relates to a medicament delivery device and to a method of using a medicament delivery device.

### BACKGROUND

Medicament delivery devices, such as auto-injectors, are known in the art for dispensing medicament to an injection site of a patient. In some cases, the needle actuator can be depressed accidentally, for example, before the device is ready and/or in position for use. Depressing the needle actuator accidentally can cause a dose of medicament to be unintentionally dispensed. This can lead to a waste of medicament.

### SUMMARY

According to an aspect of the present disclosure, there is provided a medicament delivery device for injecting medicament, wherein the medicament delivery device comprises:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a coupling configured to rotationally couple the locking member and the cap together when the cap is attached to the body, and thereby to rotationally couple the locking member to the body, so as to prevent the locking member from rotating from the pre-use position to the use position, the coupling being configured to rotationally decouple the locking member from the cap upon removal of the cap from the body so as to permit the locking member to be rotated from the pre-use position to the use position.

In some embodiments, the coupling is configured to rotationally decouple the locking member from the cap upon removal of the cap from the body in a distal direction.

In some embodiments, the coupling is configured to extend from the locking member to the cap.

In some embodiments, the locking member is provided at or towards the proximal end of the body.

In some embodiments, the locking member is configured to rotate from the pre-use position to the use position about a longitudinal axis of the body, the longitudinal axis extending from the distal end of the body to the proximal end of the body.

In some embodiments, the locking member comprises a lock ring.

In some embodiments, the actuation member is provided at or towards the proximal end of the body and, optionally, the actuation member is configured to move in a distal direction with respect to the body as the actuation member moves from the first position to the second position.

In some embodiments, the coupling is a severable coupling configured to sever or tear upon removal of the cap from the body so as to permit the locking member to be rotated from the pre-use position to the use position.

In some embodiments, the coupling comprises a severable connection configured to sever or tear upon removal of the cap from the body so as to permit the locking member to be rotated from the pre-use position to the use position.

In some embodiments, the severable connection comprises a strength discontinuity or weakness configured to sever or tear upon removal of the cap from the body.

In some embodiments, the strength discontinuity or weakness comprises a line of strength discontinuity or weakness configured to sever or tear upon removal of the cap from the body.

In some embodiments, the line of strength discontinuity or weakness is configured to extend around at least a portion of a circumference or a periphery of the coupling.

In some embodiments, the line of strength discontinuity or weakness is configured to extend around an entire circumference or periphery of the coupling.

In some embodiments, the line of strength discontinuity or weakness comprises a line of perforations.

In some embodiments, the line of strength discontinuity or weakness is provided at or towards a distal end or a proximal end of the coupling, or part-way along the longitudinal length of the coupling in a direction parallel with the longitudinal axis of the body.

In some embodiments, the coupling is configured to extend around at least a portion of a circumferential or peripheral outer surface of the body.

In some embodiments, the coupling is configured to extend entirely around a circumferential or peripheral outer surface of the body.

In some embodiments, the coupling is substantially annular or tubular, defining a cylindrical aperture extending therethrough, the body being arranged within the aperture.

In some embodiments, the body comprises a substantially cylindrical outer surface arranged within the aperture.

In some embodiments, the coupling comprises a collar arranged around the body.

In some embodiments, the collar comprises a sheet of material.

In some embodiments, the line of strength discontinuity or weakness is provided in the sheet of material.

In some embodiments, the sheet of material is provided as or comprises a label.

In some embodiments, the coupling is affixed (e.g. at a distal end thereof) to the cap so as to be rotationally coupled to the cap.

In some embodiments, the coupling is affixed to a circumferential or peripheral external surface of the cap, so as to be rotationally coupled to the cap.

In some embodiments, the coupling is adhered to the cap. In some embodiments, the coupling is adhered to a circumferential or peripheral external surface of the cap.

In some embodiments, the coupling is affixed (e.g. at a proximal end thereof) to the locking member so as to be rotationally coupled to the locking member.

In some embodiments, the coupling is affixed to a circumferential or peripheral external surface of the locking member so as to be rotationally coupled to the locking member.

In some embodiments, the coupling is adhered to the locking member. In some embodiments, the coupling is adhered to a circumferential or peripheral external surface of the locking member.

In some embodiments, the cap and the body comprise complementary locking features configured to rotationally couple the cap to the body when the cap is attached to the body.

In some embodiments, the cap is friction fitted to the body such that the cap is rotationally coupled to the body when the cap is attached to the body.

In some embodiments, the device comprises a container, such as a syringe, containing medicament.

According to another aspect of the present disclosure, there is provided a method of using a medicament delivery device for injecting medicament, the medicament delivery device comprising:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a coupling configured to rotationally couple the locking member and the cap together when the cap is attached to the body, and thereby to rotationally couple the locking member to the body, so as to prevent the locking member from rotating from the pre-use position to the use position, the coupling being configured to rotationally decouple the locking member from the cap as the cap is removed from the body so as to permit the locking member to be rotated from the pre-use position to the use position;
the method comprising:
removing the cap from the body, thereby causing the coupling to rotationally decouple the cap from the locking member so as to permit the locking member to be rotated from the pre-use position to the use position.

In some embodiments, the coupling comprises a severable connection configured to sever or tear upon removal of the cap from the body so as to permit the locking member to be rotated from the pre-use position to the use position, wherein the step of removing the cap from the body causes the severable connection to sever or tear so as to permit the locking member to be rotated from the pre-use position to the use position.

In some embodiments, the method further comprises rotating the locking member from the pre-use position to the use position.

In some embodiments, the method further comprises moving the actuation member from the first position to the second position.

According to another aspect of the present disclosure, there is provided a medicament delivery device for injecting medicament, wherein the medicament delivery device comprises:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a collar arranged external to the body, the collar being affixed to the locking member and the cap so as to rotationally couple the locking member and the cap together when the cap is attached to the body, and thereby to rotationally couple the locking member to the body, so as to prevent the locking member from rotating from the pre-use position to the use position, the collar comprising a line of strength discontinuity or weakness configured to sever or tear upon removal of the cap from the body so as to rotationally decouple the locking member from the cap and thereby permit the locking member to be rotated from the pre-use position to the use position.

In some embodiments, the collar is substantially annular or tubular, defining a substantially cylindrical aperture extending therethrough, the body being arranged within the aperture.

In some embodiments, the body comprises a substantially cylindrical outer surface arranged within the aperture.

In some embodiments, the locking member is provided at or towards the proximal end of the body.

In some embodiments, the actuation member is provided at the proximal end of the body and wherein the actuation member moves in a distal direction with respect to the body as the actuation member is moved from the first position to the second position.

In some embodiments, the line of strength discontinuity or weakness is configured to extend around at least a portion of a circumference or a periphery of the collar.

In some embodiments, the line of strength discontinuity or weakness is configured to extend around an entire circumference or periphery of the collar.

In some embodiments, the line of strength discontinuity or weakness comprises a line of perforations.

In some embodiments, the line of strength discontinuity or weakness is provided at or towards a distal end or a proximal end of the collar, or part-way along the length of the collar in a direction parallel with the longitudinal axis of the body.

In some embodiments, the collar is configured to extend around at least a portion of a circumferential or peripheral outer surface of the body.

In some embodiments, the collar is configured to extend entirely around a circumferential or peripheral outer surface of the body.

In some embodiments, the collar comprises a sheet of material.

In some embodiments, the line of strength discontinuity or weakness is provided in the sheet of material.

In some embodiments, the sheet of material is provided as or comprises a label.

In some embodiments, the collar is affixed to a circumferential or peripheral external surface of the cap so as to be rotationally coupled to the cap.

In some embodiments, the collar is adhered to the cap. In some embodiments, the collar is adhered to a circumferential or peripheral external surface of the cap.

In some embodiments, the collar is affixed to a circumferential or peripheral external surface of the locking member so as to be rotationally coupled to the locking member.

In some embodiments, the collar is adhered to the locking member. In some embodiments, the collar is adhered to a circumferential or peripheral external surface of the locking member.

In some embodiments, the cap and the body comprise complementary locking features configured to rotationally couple the cap to the body when the cap is attached to the body.

In some embodiments, the line of strength discontinuity or weakness is configured to sever or tear upon removal of the cap from the body in a distal direction.

In some embodiments, the device comprises a container, such as a syringe, containing medicament.

According to another aspect of the present disclosure, there is provided a method of using a medicament delivery device for injecting medicament, the medicament delivery device comprising:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a collar arranged external to the body, the collar being affixed to the locking member and the cap so as to rotationally couple the locking member and the cap together when the cap is attached to the body, and thereby to rotationally couple the locking member to the body, so as to prevent the locking member from rotating from the pre-use position to the use position, the collar comprising a line of strength discontinuity or weakness configured to sever or tear upon removal of the cap from the body so as to rotationally decouple the locking member from the cap and thereby permit the locking member to be rotated from the pre-use position to the use position;
the method comprising:
removing the cap from the body, thereby causing the collar to sever or tear along the line of strength discontinuity or weakness so as to rotationally decouple the cap from the locking member so as to permit the locking member to be rotated from the pre-use position to the use position.

In some embodiments, the method further comprises rotating the locking member from the pre-use position to the use position.

In some embodiments, the method further comprises moving the actuation member from the first position to the second position.

According to another aspect of the present disclosure, there is provided a method of preparing a medicament delivery device as defined in claim 1 for use prior to dispensing medicament from the device, the method comprising removing the cap from the body, thereby causing the coupling to rotationally decouple the cap from the locking member so as to permit the locking member to be rotated from the pre-use position to the use position.

According to another aspect of the present disclosure, there is provided a method of preparing a medicament delivery device as defined in claim 19 for use prior to dispensing medicament from the device, the method comprising removing the cap from the body, thereby causing the collar to sever or tear along the line of strength discontinuity or weakness so as to rotationally decouple the cap from the locking member so as to permit the locking member to be rotated from the pre-use position to the use position.

According to another aspect of the present disclosure, there is provided a method of manufacturing or assembling a medicament delivery device, wherein the medicament delivery device is defined in claim 1 or claim 19. Further optional features of the medicament delivery device are described and/or contemplated here.

In some embodiments, the method comprises attaching the cap to the body and then affixing the coupling to the cap and the locking member.

According to another aspect of the present disclosure, there is provided a method of manufacturing or assembling a medicament delivery device, wherein the medicament delivery device has the features of any of the medicament delivery devices described and/or contemplated herein.

### BRIEF DESCRIPTION OF THE DRAWINGS

Embodiments will now be described, by way of example only, with reference to the accompanying drawings, in which:

FIG. 1A is a schematic view of a medicament delivery device with a cap attached;

FIG. 1B is a schematic view of the medicament delivery device of FIG. 1A with the cap removed;

FIG. 2A is a schematic view of a medicament delivery device prior to use (i.e. in a pre-use configuration);

FIG. 2B is a schematic view of the device of FIG. 2A with the cap removed;

FIG. 2C is a schematic view of the device of FIG. 2A showing the device placed at an injection site;

FIG. 2D is a schematic view of the device of FIG. 2A with the button having been pressed to release the dispensing mechanism;

FIG. 2E is a schematic view of the device of FIG. 2A with the button having been pressed to release the dispensing mechanism;

FIG. 2F is a schematic view of the device of FIG. 2A showing the needle having retracted within the device after a dose has been delivered;

FIG. 2G is a schematic view of the device of FIG. 2A showing the device removed from the injection site after the needle has retracted within the device after delivery of the medicament;

FIG. 3A is a schematic view of a medicament delivery device prior to use (i.e in a pre-use configuration);

FIG. 3B is a schematic view of the device of FIG. 3A with the cap removed from the body of the device;

FIG. 4A is a schematic cross-sectional view of the medicament delivery device of FIG. 3A prior to use (i.e. in a pre-use configuration);

FIG. 4B is a schematic cross-sectional view of the device of FIG. 4A with the cap removed;

FIG. 4C is a schematic cross-sectional view of the device of FIG. 4A showing the device placed at an injection site;

FIG. 4D is a schematic cross-sectional view of the device of FIG. 4A with the button having been pressed to release the dispensing mechanism;

FIG. 4E is a schematic cross-sectional view of the device of FIG. 4A with the button having been pressed to release the dispensing mechanism;

FIG. 4F is a schematic cross-sectional view of the device of FIG. 4A showing the needle having retracted within the device after a dose has been delivered; and

FIG. 4G is a schematic cross-sectional view of the device of FIG. 4A showing the device removed from the injection site after the needle has retracted within the device after delivery of the medicament.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

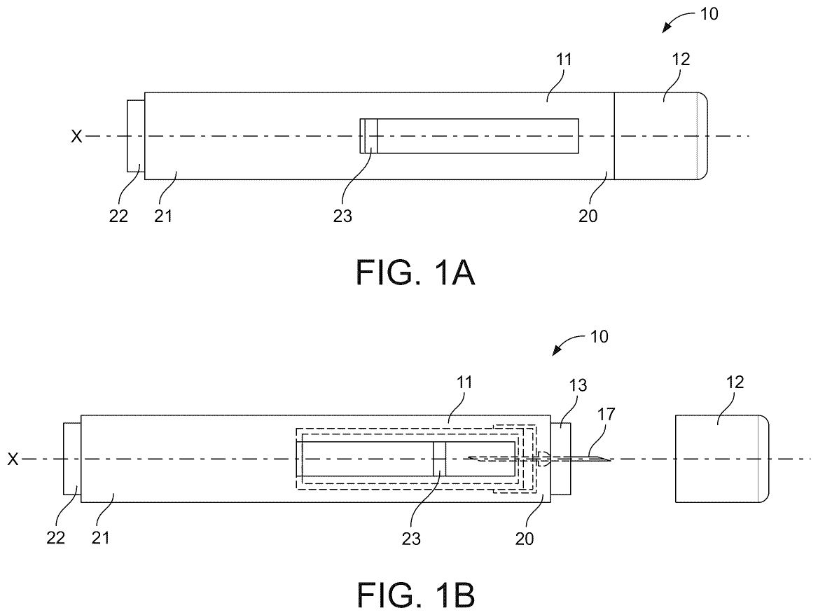

According to some embodiments of the present disclosure, an exemplary drug delivery device 10 is shown in FIGS. 1A & 1B. The device 10, as described above, is configured to inject a medicament into a patient's body. The device 10 includes a housing 11 which typically contains a reservoir containing the medicament to be injected (e.g., a syringe) and the components required to facilitate one or more steps of the delivery process. The device 10 can also include a cap assembly 12 that can be detachably mounted to the housing 11. A user removes the cap 12 from the housing 11 before device 10 is operated.

As shown, the housing 11 is substantially cylindrical and has a substantially constant diameter along the longitudinal axis X. The housing 11 has a distal region 20 and a proximal region 21. The term “distal” refers to a location that is relatively closer to a site of injection, and the term “proximal” refers to a location that is relatively further away from the injection site.

Device 10 can also include a needle sleeve 13 coupled to the housing 11 to permit movement of the sleeve 13 relative to the housing 11. For example, the sleeve 13 can move in a longitudinal direction parallel to longitudinal axis X. Specifically, movement of the sleeve 13 in a proximal direction can permit a needle 17 to extend from distal region 20 of the housing 11.

Insertion of the needle 17 can occur via several mechanisms. For example, the needle 17 may be fixedly located relative to the housing 11 and initially be located within an extended needle sleeve 13. Proximal movement of the sleeve 13 by placing a distal end of the sleeve 13 against a patient's body and moving the housing 11 in a distal direction will uncover the distal end of the needle 17. Such relative movement allows the distal end of the needle 17 to extend into the patient's body. Such insertion is termed “manual” insertion as the needle 17 is manually inserted via the patient's manual movement of the housing 11 relative to the sleeve 13.

Another form of insertion is “automated,” whereby the needle 17 moves relative to the housing 11. Such insertion can be triggered by movement of sleeve 13 or by another form of activation, for example, a button 22. As shown in FIGS. 1A & 1B, the button 22 is located at a proximal end of the housing 11. However, in other embodiments, the button 22 could be located on a side of the housing 11.

Other manual or automated features can include drug injection or needle retraction, or both. Injection is the process by which a bung or piston 23 is moved from a proximal location within a syringe (not shown) to a more distal location within the syringe in order to force a medicament from the syringe through the needle 17. In some embodiments, a drive spring (not shown) is under compression before the device 10 is activated. A proximal end of the drive spring can be fixed within proximal region 21 of the housing 11, and a distal end of the drive spring can be configured to apply a compressive force to a proximal surface of the piston 23. Following activation, at least part of the energy stored in the drive spring can be applied to the proximal surface of piston 23. This compressive force can act on piston 23 to move it in a distal direction. Such distal movement acts to compress the liquid medicament within the syringe, forcing it out of the needle 17.

Following injection, the needle 17 can be retracted within the sleeve 13 or housing 11. Retraction can occur when the sleeve 13 moves distally as a user removes the device 10 from a patient's body. This can occur as the needle 17 remains fixedly located relative to the housing 11. Once a distal end of the sleeve 13 has moved past a distal end of needle 17, and the needle 17 is covered, the sleeve 13 can be locked. Such locking can include locking any proximal movement of the sleeve 13 relative to the housing 11.

Another form of needle retraction can occur if the needle 17 is moved relative to the housing 11. Such movement can occur if the syringe within the housing 11 is moved in a proximal direction relative to the housing 11. This proximal movement can be achieved by using a retraction spring (not shown), located in the distal region 20. A compressed retraction spring, when activated, can supply sufficient force to the syringe to move it in a proximal direction. Following sufficient retraction, any relative movement between the needle 17 and the housing 11 can be locked with a locking mechanism. In addition, the button 22 or other components of the device 10 can be locked as required.

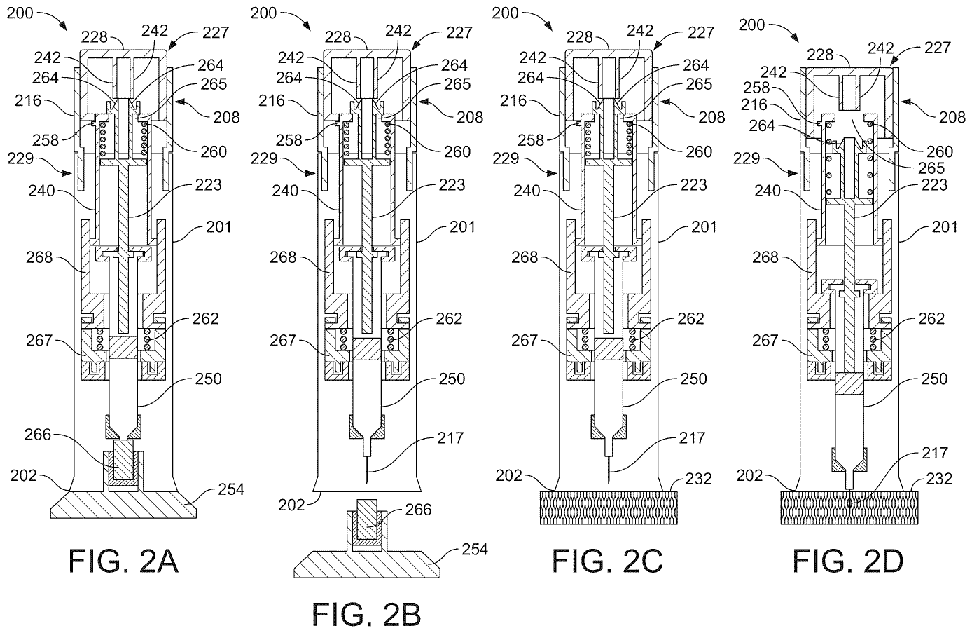

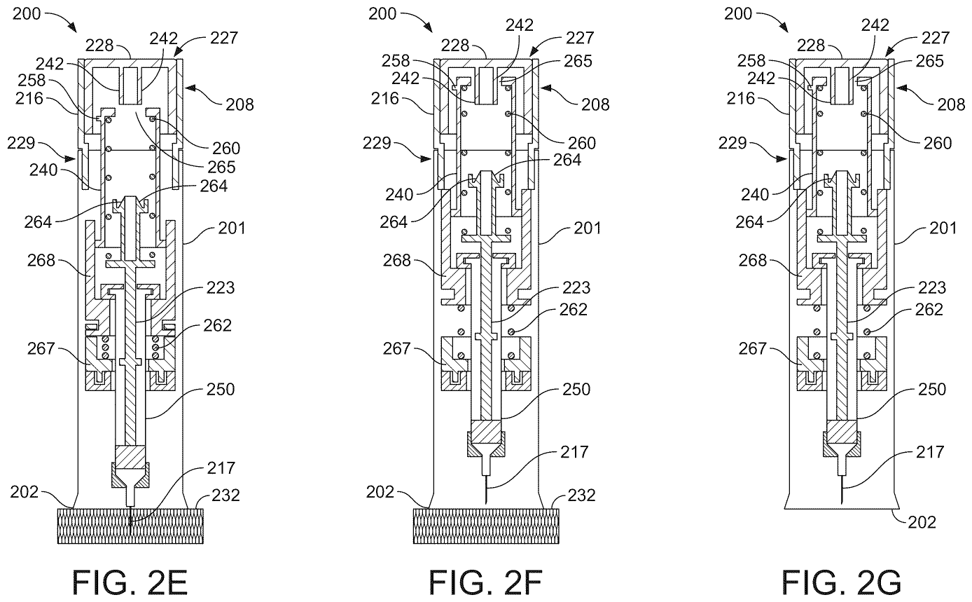

FIGS. 2A to 2G show the sequential steps of operating a medicament delivery device 200. The medicament delivery device 200 is an autoinjector.

The device 200 comprises a body 201, a syringe 250 having a needle 217 and an axially moveable plunger 223 for dispensing medicament from the syringe 250. The device comprises a cap 254 which is removably attached to the body 201 and covers a distal end 202 of the body 201 for preventing access to the needle 217. The device 200 has a needle shield 266 that covers the needle 217 before use. The needle shield 266 is attached to the cap 254.

The medicament delivery device 200 has a dispensing mechanism 229. The medicament delivery device 200 has an actuation member 227 which is configured to release the dispensing mechanism 229. The actuation member 227 is configured to engage the dispensing mechanism 229 to release the dispensing mechanism 229.

The dispensing mechanism 229 is further configured to cause the needle 217 to move distally from a needle pre-use position, in which the needle 217 is recessed within the body 201, to an injection position in which the needle 217 protrudes from the distal end 202 of the body 201 when the dispensing mechanism 229 is released.

The dispensing mechanism 229 is configured to dispense the medicament from the needle 217 when the needle 217 is in the injection position.

As shown in FIGS. 2B-2C, in order to deliver a dose of medicament to an injection site, the cap 254 is removed (FIG. 2B) and the device is placed at an injection site 232 (FIG. 2C).

The actuation member 227 comprises a button 228 and is prevented from being depressed by a stop 258. The stop is provided on the spring guide 240, for example.

The device has a locking member 208 in the form of a lock ring 216 which is rotatable by a user about a longitudinal axis of the device. The actuation member 227 is keyed to the lock ring 216 so that the actuation member 227 rotates with the lock ring 216. The lock ring 216 is rotatable from a pre-use position, in which distal movement of the button 228 is prevented, to a use position in which distal movement of the button 228 is permitted.

When the lock ring 216 is in the pre-use position then the stop 258 engages the button 228 to prevent the button 228 from being depressed.

In order to allow the button 228 to be depressed, the lock ring 216 is rotated about the longitudinal axis of the device from the pre-use position to the use position. The rotation of the lock ring 216 also rotates the actuation member 227 to a position in which the stop 258 no longer prevents the button 228 from being depressed as shown, for example, in FIG. 2C.

Turning now to FIG. 2D, the user then presses the button 228 to release the dispensing mechanism 229 for dispensing medicament from the device. The dispensing mechanism 229 has a plunger 223 and a bias in the form of a compression spring 260. The plunger 223 is biased distally by the spring 260.

The dispensing mechanism 229 is at least partially housed within the spring guide 240. The plunger 223 has a release member which has proximally-extending clips 264. The spring 260 is retained in the compressed position by virtue of the clips 264 which protrude through a proximal opening 265 in the spring guide 240. The clips 264 engage the spring guide 240 for maintaining the plunger 223 in a proximal position.

The actuation member 227 has a firing member comprising a pair of protrusions 242 which engage with the clips 264 when the button 228 is depressed to flex the clips 264 radially inwardly thereby allowing the clips 264 to move distally through the proximal opening 265 to release the spring 260.

When the dispensing mechanism 229 is released, then the syringe 250 is released for distal axial movement towards the injection site 232 such that the needle 217 moves from the needle pre-use retracted position to an exposed (or “uncovered” or “injection”) position for delivering medicament to the injection site 232 under the biasing force of the compression spring 260.

Depressing the button 228 releases the plunger 223 which, biased by the bias 260, moves along the syringe 250 towards the distal end of the device 200 to force medicament within the syringe 250 through the needle 217, thereby delivering a dose of medicament as shown, for example in FIG. 2E.

As shown in FIG. 2F, once the dose of medicament has been delivered, a medicament container bias 262, embodied by a further spring 262, then causes the needle 217 to move axially back to the retracted position, away from the injection site 232 in a proximal direction. The plunger 223 flexes a clip (not shown) on a first collar 267 which allows the first collar 267 to rotate relative to the body 201 and relative to a second collar 268. The first collar 267 rotates from a first position in which the second collar 268 is axially coupled to the first collar 267, into a second position in which the second collar 268 is free to move axially relative to the first collar 267. For example, the second collar 268 may comprise a radially protruding coupling element configured to be received in or engage with a corresponding receiving portion of the first collar 267, such that rotating the first collar 267 from the first position into the second position causes the coupling element to be moved out from the receiving portion, to allow the second collar 268 to move axially relative to the first collar 267. Axial movement of the second collar 268 permits the needle 217 to be retracted.

As shown in FIG. 2G, the device 200 is then removed from the injection site 232, for disposal.

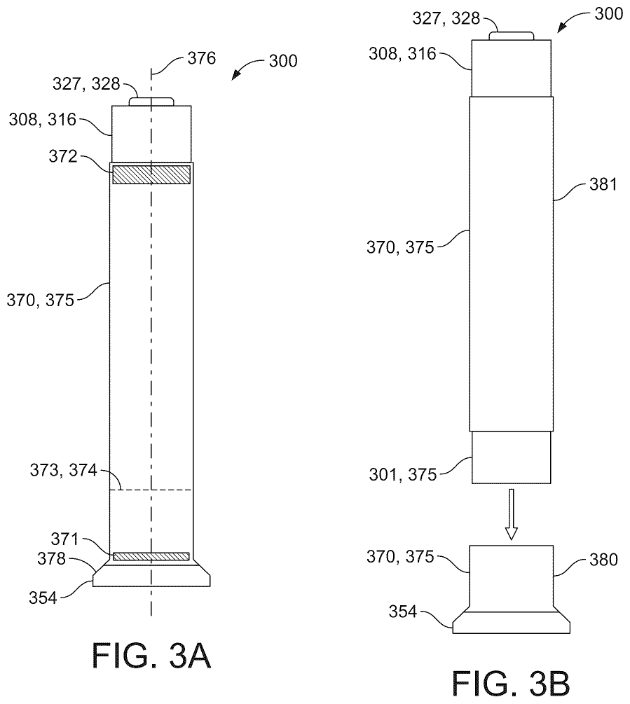

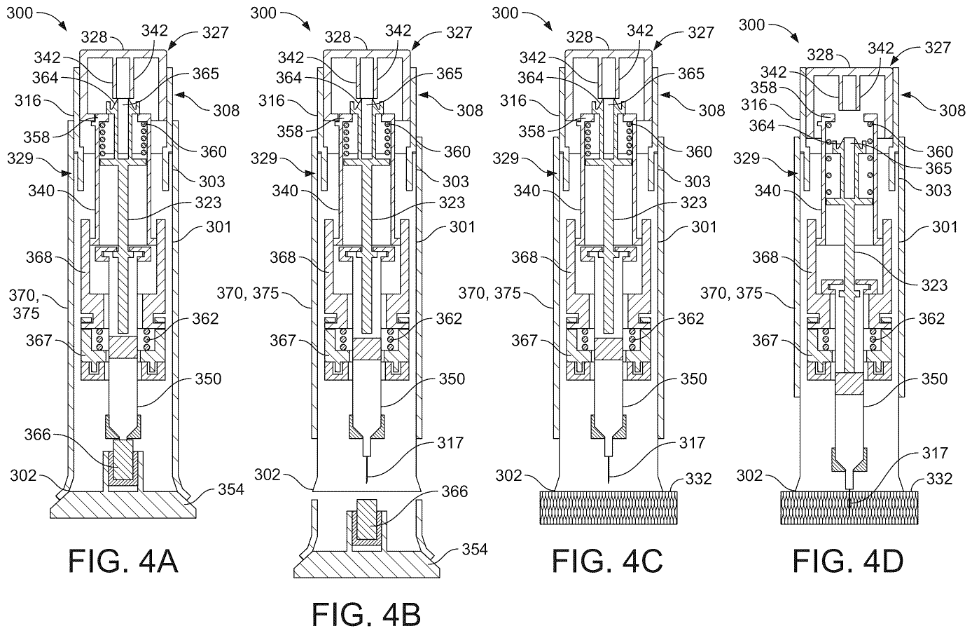

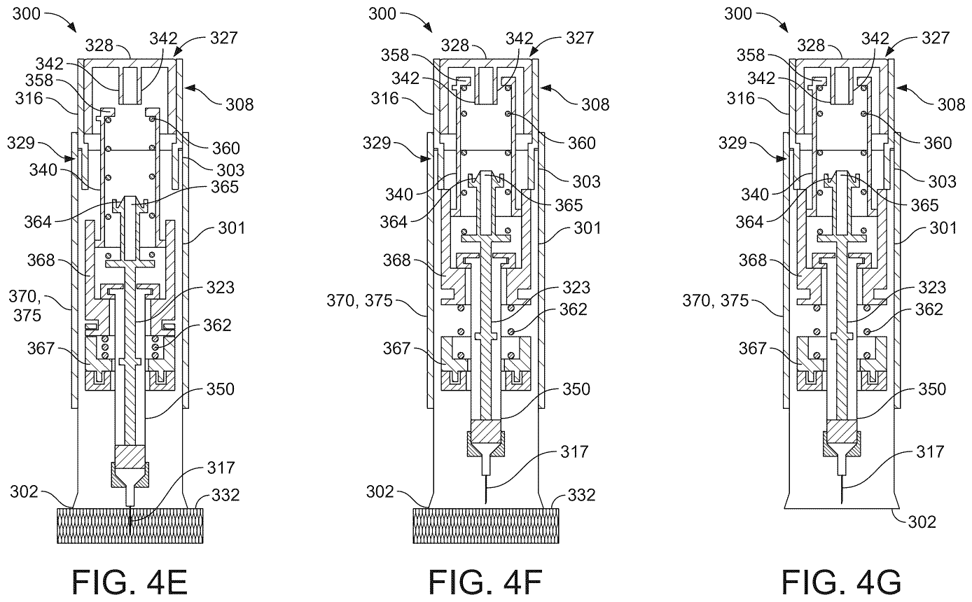

FIGS. 3A and 3B and FIGS. 4A to 4G are schematic views of parts of a medicament delivery device 300 in accordance with an embodiment of the disclosure. The medicament delivery device 300 is substantially the same as the medicament delivery device 200 of FIG. 2A to 2G and so corresponding features of the medicament delivery device 300 of FIGS. 3A and 3B and FIGS. 4A to 4G and the medicament delivery device 200 of FIGS. 2A to 2G share corresponding reference numerals, with the reference numerals of the medicament delivery device 300 of FIGS. 3A and 3B and FIGS. 4A to 4G being increased by 100 as compared to the corresponding reference numerals of the medicament delivery device 200 of FIGS. 2A to 2G. Discussion features of the medicament delivery device 300 of FIGS. 3A and 3B and FIGS. 4A to 4G which correspond to features of the embodiment of FIGS. 2A to 2G is omitted for the sake of brevity.

As is common with the medical delivery device 200 of FIG. 2A to 2G, the actuation member 327 and locking member 308 are provided at a proximal end 303 of the body 301. The actuation member 327 is movable in a distal direction with respect to the body 301 from a first position to a second position for dispensing medicament from the needle 317.

As best shown in FIGS. 3A and 3B, the medicament delivery device 300 comprises a coupling 375. The coupling 375 is configured to rotationally couple the locking member 308 and the cap 354 together when the cap 354 is attached to the body 301. The cap 354 is rotationally coupled to the body 301 when the cap 354 is attached to the body 301, for example by the cap 354 being friction fitted to the body 301 or by complementary locking features (not shown), for example one of the cap 354 and the body 301 may comprise two diametrically opposed guide pins each slidably received within a respective axially extending slot provided in the other of the cap 354 or the body 301. Thus, the coupling 375 rotationally couples the locking member 308 to the body 301 via the cap 354 so as to prevent the locking member 308 from rotating (or being rotated, e.g. by a user) from the pre-use position to the use position. The coupling 375 is configured to rotationally decouple the locking member 308 from the cap 354 as the cap 354 is removed from the body 301. Thus, after the cap 354 has been removed from the body 301, the locking member 308 is free to be rotated (e.g. by a user) from the pre-use position to the use position.

The coupling 375 in this embodiment is provided as a collar 370 (or sleeve 370) which extends around an entire circumferential outer surface 377 of the body 301, although other embodiments in which the collar 370 extends around only a portion of the outer surface 377 of the body 301 are also envisaged. Thus, the collar 370 is substantially tubular in shape, defining a cylindrical aperture extending therethrough in which a substantially cylindrical outer surface 377 of the body 301 is arranged. The coupling 375 extends from the locking member 308 to the cap 354. The collar 370 in this embodiment comprises a sheet of material, for example of a substantially constant thickness, and in some embodiments the collar 370 may be provided as a label, for example with printing thereon. In some embodiments, the sheet of material may be substantially flexible or in other embodiments the sheet of material may be substantially rigid. In some embodiments the coupling 375 or collar 370 may not be (e.g. directly) rotationally coupled, e.g. affixed or attached, to the body 301 when the cap 354 is attached to the body 301, for example the coupling 375 or collar 370 may not be directly affixed or attached to the body 301 when the cap 354 is attached to the body 301 so as to be rotationally decoupled from the body 301 when the cap 354 is attached to the body 301.

The coupling 375 extends from the locking member 308 to the cap 354 such that a distal portion of the coupling 375 overlaps a portion of the cap 354 and a proximal portion of the coupling 375 overlaps a portion of the locking member 308. The coupling 375 is affixed at a distal end thereof to the cap 354 by a first adhesive region 371, arranged between the overlapping distal portion of the coupling 375 and a peripheral or circumferential external surface 378 of the cap 354, and the coupling 375 is affixed at a proximal end thereof to the locking member 308 by a second adhesive region 372, arranged between the overlapping proximal portion of the coupling 375 and a peripheral or circumferential external surface 379 of the locking member 308. Thus, the first adhesive region 371 adheres the distal end of the coupling 375 to the cap 354 and the second adhesive region 372 adheres the proximal end of the coupling 375 to the locking member 308. Any other suitable means for affixing the coupling 375 to the cap 354 and locking member 308 may instead be used, for example in some embodiments the distal and proximal portions of the coupling 375 may be heat sealed to the cap 354 and the locking member 308 respectively.

The coupling 375 of this embodiment is a severable coupling in that it is configured to sever (i.e. divide in two, e.g., into a distal portion 380 which is affixed to the cap 354 and a proximal portion 381 which is affixed to the locking member 308) or tear upon removal of the cap 354 from the body 301 so as to permit the locking member 308 to be rotated from the pre-use position to the use position. The severable coupling 375 comprises a severable connection 373 which comprises a strength discontinuity or weakness. In this embodiment, the severable connection 373 comprises a peripherally extending line 374 of strength discontinuity or weakness along which the coupling 375 is configured to sever or tear upon removal of the cap 354. The line 374 of strength discontinuity or weakness in this embodiment comprises a line 374 of perforations in the sheet of material of the collar 370, although any other suitable severable connection 373, or line 374 of strength discontinuity or weakness, may instead be used, for example the line 374 of strength discontinuity or weakness may in some embodiments comprise a line 374 of reduced thickness (i.e. of reduced wall thickness of the sleeve or collar 370).

The line 374 may extend around a portion of the periphery of the coupling 375 or the line 374 may, as in this embodiment, extend entirely around the periphery of the coupling 375. The line 374 in this embodiment is a straight line which lies in a plane which is substantially perpendicular to the longitudinal axis 376 of the body 301, although this is not essential and any other orientations and configurations of the line 374 may instead be used, for example in some embodiments the line 374 may lie in a plane which is angled at an oblique angle to the longitudinal axis 376 of the body 301 and/or in some embodiments the line 374 may be curvilinear.

In this embodiment, the line 374 is provided towards the distal end of the coupling 375, although the line 374 may instead be provided at any other suitable position, for example at the distal end of the coupling 375, at or towards a proximal end of the coupling 375, or between the distal end and the proximal end of the coupling 375 so as to be provided part-way along the longitudinal length of the coupling 375.

FIG. 3B illustrates the removal of the cap 354 causing the severable connection 373 of the coupling 375 (in this embodiment, the line of perforations 374) to sever or tear (and thereby cause the severable coupling 375 to divide the coupling 375 into a distal portion 380 which is affixed to the cap 354 and a proximal portion 381 which is affixed to the locking member 308) so as to rotationally decouple the locking member 308 from the body 301 to thereby permit the locking member 308 to be rotated from the pre-use position to the use position.

FIGS. 4A to 4G show the sequential steps of operating the medicament injection device 300. FIGS. 4A and 4B correspond to the views of FIGS. 3A and 3B.

FIG. 4A shows the device in the pre-use configuration in which the cap 354 is attached to a distal end 302 of the body 301 and the locking member 308 is in the pre-use position in which the actuation member 327 is prevented from moving distally from a first position to a second position, for example when a user depressed the button 328.

As shown in FIG. 4B, the cap 354 is first removed from the body 301, for example by a user, by moving the cap 354 in a distal direction away from the distal end 302 of the body 301. The removal of the cap 354 causes the coupling 375 to rotationally decouple the locking member 308 from the cap 354 by causing the coupling 375 to sever or tear along the line of strength discontinuity or weakness 374, thereby severing the severable connection 373. Thus, the locking member 308 is now free to be rotated, for example by a user, from the pre-use position to the use position. The subsequent steps shown in FIGS. 4C to 4G are then identical to those discussed above with reference to FIGS. 2C to 2G.

The medicament delivery devices described herein may have some or all of the features as described in relation to the medicament delivery device 200.

The dispensing mechanism 229 may have the some or all of the features as described and/or contemplated in relation to FIGS. 2A to 2G.

In another embodiment, the dispensing mechanism may have alternative or additional features to those described and/or contemplated in relation to FIGS. 2A to 2G. The dispensing mechanism may have features as described and/or contemplated herein, for example in relation to FIGS. 1A and 1B.

The dispensing mechanism provides one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

The medicament delivery device can include various types of safety syringe, pen-injector, or auto-injector. The device can include a cartridge-based system that requires piercing a sealed ampule before use.

Distal movement of the actuation member may cause automatic dispensing of the medicament from the device and/or distal movement of the actuation member may cause the distal movement of the needle from a needle pre-use position to a needle injection position. The dispensing mechanism may be configured to dispense medicament from the needle when the dispensing mechanism is released.

In the needle pre-use position the needle may be flush with the distal end of the body or the needle may be recessed within the body. In another embodiment the needle may be fixed in position relative to the body.

In another device, different features may be provided to prevent the actuation member from moving distally. For example, the stop may be provided on another component of the medicament delivery device. In another device a lock ring 216 is not present.

### LIST OF FEATURES

10—Device
11—housing
12—cap
13—needle sleeve
17—needle
20—distal region
21—proximal region
22—button
23—piston
200—medicament delivery device
201—body
202—distal end of the body
208—locking member
216—lock ring
217—needle
223—plunger
227—actuation member
228—button
229—dispensing mechanism
232—injection site
240—spring guide
242—protrusions
250—syringe
254—cap
258—stop
260—spring
262—spring
264—clip
265—proximal opening
266—needle shield
267—collar
268—collar
300—medicament delivery device
301—body
302—distal end of the body
303—proximal end of the body
308—locking member
316—lock ring
317—needle
323—plunger
327—actuation member
328—button
329—dispensing mechanism
332—injection site
340—spring guide
342—protrusions
350—syringe
354—cap
358—stop
360—spring
362—spring
364—clip
365—proximal opening
366—needle shield
367—collar
368—collar
370—collar
371—first adhesive region
372—second adhesive region
373—severable connection
374—line
375—coupling
376—longitudinal axis
377—outer surface of body
378—outer surface of the cap
379—external surface of the locking member
380—distal portion
381—proximal portion

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, small modular immunopharmaceuticals (SMIP), binding-domain immunoglobulin fusion proteins, camelized antibodies, and immunoglobulin single variable domains. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. As such, immunoglobulin single variable domains are capable of specifically binding to an epitope of the antigen without pairing with an additional immunoglobulin variable domain. The binding site of an immunoglobulin single variable domain is formed by a single heavy chain variable domain (VH domain or VHH domain) or a single light chain variable domain (VL domain). Hence, the antigen binding site of an immunoglobulin single variable domain is formed by no more than three CDRs.

An immunoglobulin single variable domain (ISV) can be a heavy chain ISV, such as a VH (derived from a conventional four-chain antibody), or VHH (derived from a heavy-chain antibody), including a camelized VH or humanized VHH. For example, the immunoglobulin single variable domain may be a (single) domain antibody, a “dAb” or dAb or a Nanobody® ISV (such as a VHH, including a humanized VHH or camelized VH) or a suitable fragment thereof. [Note: Nanobody® is a registered trademark of Ablynx N.V.]; other single variable domains, or any suitable fragment of any one thereof.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to WO 2004/068820, WO 2006/030220, WO 2006/003388. It should also be noted that, although less preferred in the context of the present invention because they are not of mammalian origin, single variable domains can be derived from certain species of shark (for example, the so-called “IgNAR domains”, see for example WO 2005/18629).

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example of a compound to be administered with the drug delivery device disclosed herein is a compound with the INN tirzepatide, as referenced in claim 1 of U.S. Pat. No. 9,474,780.

An example of a pharmaceutical composition to be administered with the drug delivery device disclosed herein is a pharmaceutical composition as referenced in U.S. Pat. No. 11,357,820.

An example of a pharmaceutical composition to be administered with the drug delivery device disclosed herein includes a 0.5 mL solution of 2.5 mg, 5 mg, 7.5 mg, 10 mg, 12.5 mg, or 15 mg of tirzepatide and the following excipients sodium chloride (4.1 mg), sodium phosphate dibasic heptahydrate (0.7 mg), and water for injection. Hydrochloric acid solution and/or sodium hydroxide solution may be added to adjust the pH.

An example starting dosage tirzepatide may be 2.5 mg injected subcutaneously once weekly. After four weeks, the tirzepatide dosage may be increased to 5 mg injected subcutaneously once weekly. The dosage may be further increased in 2.5 mg increments after at least four weeks on the current dose. In an example, the maximum dosage of tirzepatide may be 15 mg injected subcutaneously once weekly. If a dose is missed, patients may be instructed to administer tirzepatide as soon as possible within four days (96 hours) after the missed dose. If more than four days have passed, patients may skip the missed dose and administer the next dose on the regularly scheduled day. In each case, patients may then resume their regular once weekly dosing schedule. The day of weekly administration may be changed, if necessary. The time between two doses may be at least three days (72 hours).

Tirzepatide dosages may include 2.5 mg/0.5 mL, 5 mg/0.5 mL, 7.5 mg/0.5 mL, 10 mg/0.5 mL, 12.5 mg/0.5 mL, and 15 mg/0.5 mL. Tirzepatide may be stored in a refrigerator at 2° C. to 8° C. (36° F. to 46° F.). A single-dose pen or single-dose vial may be stored unrefrigerated at temperatures not to exceed 30° C. (86° F.) for up to 21 days. Tirzepatide may be stored in a carton.

1-20. (canceled)

21. A medicament delivery device for injecting medicament, wherein the medicament delivery device comprises:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body, the cap is rotationally coupled to the body and covers the distal end of the body for preventing access to the needle; and
a coupling configured to rotationally couple the locking member and the cap together when the cap is attached to the body, and thereby to rotationally couple the locking member to the body, so as to prevent the locking member from rotating from the pre-use position to the use position, the coupling being configured to rotationally decouple the locking member from the cap upon removal of the cap from the body so as to permit the locking member to be rotated from the pre-use position to the use position,
wherein the coupling comprises a distal portion affixed to the cap and a proximal portion affixed to the locking member, and
wherein the coupling comprises a severable connection positioned between the distal portion and the proximal portion, wherein the severable connection is configured to sever or tear upon removal of the cap from the body to permit the locking member to be rotated from the pre-use position to the use position, and wherein the distal portion and the proximal portion are configured to separate from each other when the severable connection being severed or torn.

22. The medicament delivery device according to claim 21, wherein the locking member is provided at or towards the proximal end of the body.

23. The medicament delivery device according to claim 21, wherein the actuation member is provided at the proximal end of the body and the actuation member is configured to move in a distal direction with respect to the body as the actuation member moves from the first position to the second position.

24. (canceled)

25. The medicament delivery device according to claim 21, wherein the severable connection comprises a strength discontinuity or weakness configured to sever or tear upon removal of the cap from the body.

26. The medicament delivery device according to claim 25, wherein the strength discontinuity or weakness comprises a line of strength discontinuity or weakness configured to sever or tear upon removal of the cap from the body.

27. The medicament delivery device according to claim 26, wherein the line of strength discontinuity or weakness is configured to extend around an entire circumference or periphery of the coupling.

28. The medicament delivery device according to claim 26, wherein the line of strength discontinuity or weakness comprises a line of perforations.

29. The medicament delivery device according to claim 21, wherein the coupling is configured to extend around at least a portion of a circumferential or peripheral outer surface of the body

30. The medicament delivery device according to claim 29, wherein the coupling is configured to extend entirely around a circumferential or peripheral outer surface of the body.

31. The medicament delivery device according to claim 30, wherein the coupling comprises a collar arranged around the body.

32. The medicament delivery device according to claim 31, wherein the collar comprises a sheet of material.

33. The medicament delivery device according to claim 21, wherein the coupling is affixed to the cap so as to be rotationally coupled to the cap.

34. The medicament delivery device according to claim 33, wherein the coupling is adhered to the cap.

35. The medicament delivery device according to claim 21, wherein the coupling is affixed to the locking member so as to be rotationally coupled to the locking member.

36. The medicament delivery device according to claim 35, wherein the coupling is adhered to the locking member.

37. The medicament delivery device according to claim 21, wherein the medicament delivery device comprises a container containing medicament.

38. A method of using a medicament delivery device for injecting medicament, the medicament delivery device comprising:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a coupling configured to rotationally couple the locking member and the cap together when the cap is attached to the body, and thereby to rotationally couple the locking member to the body, so as to prevent the locking member from rotating from the pre-use position to the use position, the coupling being configured to rotationally decouple the locking member from the cap as the cap is removed from the body so as to permit the locking member to be rotated from the pre-use position to the use position,
wherein the coupling comprises a distal portion affixed to the cap and a proximal portion affixed to the locking member, and
wherein the coupling comprises a severable connection positioned between the distal portion and the proximal portion, wherein the severable connection is configured to sever or tear upon removal of the cap from the body to permit the locking member to be rotated from the pre-use position to the use position, and wherein the distal portion and the proximal portion are configured to separate from each other when the severable connection is severed or torn;
the method comprising:
removing the cap from the body, thereby causing the coupling to rotationally decouple the cap from the locking member so as to permit the locking member to be rotated from the pre-use position to the use position.

39. The method according to claim 38, wherein the step of removing the cap from the body causes the severable connection to sever or tear so as to permit the locking member to be rotated from the pre-use position to the use position.

40. A medicament delivery device for injecting medicament, wherein the medicament delivery device comprises:
a body having a proximal end and an opposed distal end;
a needle for injecting medicament;
an actuation member movable relative to the body from a first position to a second position for dispensing medicament from the needle;
a locking member configured to rotate relative to the body from a pre-use position, in which the actuation member is prevented from moving to the second position, to a use position in which the actuation member is permitted to move to the second position;
a cap removably attached to the body, wherein when the cap is attached to the body then the cap covers the distal end of the body for preventing access to the needle, the cap being rotationally coupled to the body when the cap is attached to the body; and
a collar arranged external to the body, the collar comprising a proximal portion affixed to the locking member and a distal portion affixed to the cap so as to rotationally couple the locking member and the cap together when the cap is attached to the body, and thereby to rotationally couple the locking member to the body, so as to prevent the locking member from rotating from the pre-use position to the use position, the collar comprising a line of strength discontinuity or weakness configured to sever or tear upon removal of the cap from the body so as to rotationally decouple the locking member from the cap and thereby permit the locking member to be rotated from the pre-use position to the use position,
wherein the line of strength discontinuity or weakness is positioned between the distal portion and the proximal portion, and wherein the distal portion and the proximal portion are configured to separate from each other when the line of strength discontinuity or weakness is severed or torn.
